(19) United States
(12) Patent Application Publication
(10) Pub. No.: US 2023/0357610 A1
(43) Pub. Date: Nov. 9, 2023

(54) PRESSURE-SENSITIVE ADHESIVES AND RELATED METHODS

(71) Applicants: Shawn Swanson, Seattle, WA (US); Alshakim Nelson, Seattle, WA (US); John Devin MacKenzie, Seattle, WA (US); UNIVERSITY OF WASHINGTON, Seattle, WA (US)

(72) Inventors: Eric J. Seibel, Seattle, WA (US); Leonard Y. Nelson, Seattle, WA (US); Christopher R. Fellin, Seattle, WA (US); Shawn Swanson, Seattle, WA (US); Alshakim Nelson, Seattle, WA (US); John Devin MacKenzie, Seattle, WA (US)

(73) Assignee: UNIVERSITY OF WASHINGTON, Seattle, WA (US)

(21) Appl. No.: 18/044,474

(22) PCT Filed: Sep. 9, 2021
(86) PCT No.: PCT/US2021/049718

§ 371 Date: Mar. 8, 2023

Related U.S. Application Data
(60) Provisional application No. 63/075,946, filed on Sep. 9, 2020.

(51) Int. Cl. C09J 133/08; C09J 7/38; C09J 7/25

(52) U.S. Cl. CPC C09J 133/08; C09J 7/385; C09J 7/255; C09J 2301/502; C09J 2301/302

(57) ABSTRACT

Temperature-responsive pressure sensitive adhesives and articles of manufacture, such as bandages and medical tape, incorporating the adhesives are described. Methods for selection of a class of pressure sensitive adhesives for embedding a temperature sensitive polymer into the adhesive formulations are also provided.

### CROSS-REFERENCE TO RELATED APPLICATION

This application claims the benefit of U.S. Patent Application No. 63/075,946, filed Sep. 9, 2020, the contents of which are hereby incorporated by reference in their entirety.

### STATEMENT OF GOVERNMENT RIGHTS

This invention was made with government support under Grant No. DMR-1752972 awarded by the National Science Foundation and under Grant No. U01 HL152401 awarded by the National Institutes of Health. The government has certain rights in the invention.

### BACKGROUND

Medical tapes and bandages consist of a backing material, for example polyethylene terephthalate (PET), that is coated with a pressure sensitive adhesive (PSA). These adhesives have sufficient tack such that only light pressure is required for application to skin. Adhesives are considered Class I, low risk medical devices by the FDA. The adhesive serves to prevent ingression of contaminants into a wound. Often the adhesive will secure an intravenous (IV) line to a patient's skin. Injury to the skin during removal of the tape is a common problem for geriatric and young pediatric patients. Skin damage can also occur for patients using blood thinners or steroids. Medical Adhesive-Related Skin Injuries (MARSI) not only damage the skin integrity, but also causes pain during removal, exposes the patient to a higher risk of infection and may enlarge the wound size and extend the time for healing.

Softening of adhesion by adding solvent is a common method for reducing the adhesion between a medical tape and skin. However, this process is slow and puts significant tension on the skin or critical device when trying to expose more of the binding adhesive by pulling up on the tape. Raising temperature is another method of softening sticky adhesives, but the maximum temperature range is limited by the pain threshold. The change in adhesion must be created in a narrow temperature range for human skin so that high adhesion is maintained for fever conditions while staying below the skin pain threshold.

Silicone based adhesives offer lower peel force properties. However, these adhesives often fail to securely hold an IV in place for an extended time. Accidental removal of an IV line causes additional patient trauma and requires hospital staff intervention.

Embedding additives into pressure sensitive adhesives (PSAs) to mitigate the adhesive bonding to the skin has been demonstrated. However, the incompatibility of some PSA systems with the additive required many additional steps to remove some of the solvent and replace it with solvents to improve additive solubility. This cumbersome step adds cost and complexity to the fabrication process. It may also introduce other problems during the PSA drying cycle when the solvents are evaporated. PSA solvent blends are carefully tailored to prevent pinholes in the PSA coating during the drying step. Replacing the solvents may result in an increased propensity of the PSA to form pinholes. Additives with functionality, e.g. acrylic acid adducts, or copolymers with shorter chain acrylates, have been disclosed. However, these modifications to the additive introduce other problems such as reactivity with PSA crosslinking mechanisms that improve adhesive cohesion or sub-optimal melting temperatures. Further, typically the adhesive is a soft polymer matrix that contains a tackifier that increases the tack or stickiness of the surface of the PSA upon contact. The PSA holds more strongly onto the substrate when applied with pressure. A tape that has temperature-sensitive adhesive properties can be obtained commercially, see Intelimer® Tape, manufactured by Nitta Corporation (Osaka, Japan) under license from the Landec Corporation (Menlo Park, CA). However, the transition temperature from high to low adhesion is above 45° C., which is too high of temperature for human skin application.

Therefore, a need exists for PSA formulations and solvent blends compatible with an embedded additive that do not have reactive side chains or sub-optimal melting temperatures.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

This disclosure relates to pressure sensitive adhesives and articles of manufacture, such as bandages and medical tape, incorporating the adhesives. Methods for selection of a class of pressure sensitive adhesives for embedding a temperature sensitive polymer into the adhesive formulations are also provided, as well as methods of manufacturing a PSA with additives. Peel force is also defined, with switchable peel forces enabled by the PSA and additive combinations.

In one aspect, a pressure sensitive adhesive (PSA) comprising (1) a polymeric pressure-sensitive adhesive component, and (2) a temperature responsive additive to the polymeric PSA component is disclosed.

In another aspect, an article of manufacture comprising an adhesive composition is disclosed.

In yet another aspect, a switchable tape comprised of a tape backing later and an adhesive layer, wherein the adhesive layer further comprises an adhesive dropping in peel force upon exposure to temperature over 35° C. is disclosed.

In another aspect, a method of reducing adhesive peel force at elevated temperatures of an adhesive composition comprising an acrylic-rubber-hybrid, the method comprising adding to the solvent borne adhesive composition about 0.1 wt % to about 6 wt % of a crystalline polymeric additive to the composition, wherein the crystalline polymeric additive is selected from methacrylate or acrylate polymer comprising monomeric units of formula I:

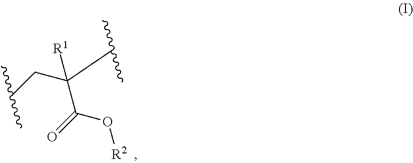

wherein R¹ is H or CH₃, and R² is a C₁₂-C₂₀ linear alkyl is disclosed.

In yet another aspect, a method of using a composition comprising applying heat to the composition and removing the composition from a surface is disclosed.

In another aspect, a PSA comprising a polymeric pressure-sensitive adhesive component, and a temperature sensitive additive, and wherein the temperature sensitive additive forms phase separated nanometer-sized domains is disclosed.

In yet another aspect, a method of making a pressure sensitive adhesive composition, the method comprising mixing a solvent-borne PSA with a temperature responsive additive, applying the mixed adhesive and additive to a surface to be adhered, and drying the adhesive is disclosed.

In another aspect, a method of making a temperature-switchable adhesive, the method comprising applying a PSA to a surface to be adhered, drying the adhesive, and printing a temperature responsive additive onto the adhesive is disclosed.

In another aspect, a method of making a temperature-switchable adhesive, the method comprising any combination of grooving or embossing a first PSA surface, and adding an additional layer of PSA with temperature responsive additives dissolved in a solvent mixture that is sprayed, coated printed, or laid over the first PSA surface is disclosed.

### DESCRIPTION OF THE DRAWINGS

The foregoing aspects and many of the attendant advantages of claimed subject matter will become more readily appreciated as the same become better understood by reference to the following detailed description, when taken in conjunction with the accompanying drawings, wherein:

FIG. 1 is an illustration of an example adhesive composition in accordance with the present technology;

FIG. 2 is an illustration of the formation of example nanometer-sized domains in an example adhesive composition in accordance with the present technology;

FIGS. 3A-B are representative tapping mode atomic force microscopy (AFM) images of example nanometer-sized domains in accordance with the present technology;

FIG. 4 is a graph of reduction in adhesion measured using a temperature-controlled peel force tester in accordance with the present technology;

FIGS. 5A-B are illustrations of example fabrication processes of an example temperature-responsive medical tape “UnTape”, in accordance with the present technology;

FIGS. 6A-B are diagrams of a flexographic printing plate transferring additive ink domains to the adhesive surface in a defined pattern in accordance with the present technology;

FIGS. 7A-C shows a series in time that describes an example mechanism of release from temperature-responsive domains on the surface of the PSA, which can be in contact with skin in accordance with the present technology; and

FIG. 8A-D illustrate alternative methods of creating a higher concentration of temperature-responsive additive at or near the surface of the PSA in accordance with the present technology.

### DETAILED DESCRIPTION

The detailed description set forth below in connection with the appended drawings, where like numerals reference like elements, is intended as a description of various embodiments of the disclosed subject matter and is not intended to represent the only embodiments. Each embodiment described in this disclosure is provided merely as an example or illustration and should not be construed as preferred or advantageous over other embodiments. The illustrative examples provided herein are not intended to be exhaustive or to limit the claimed subject matter to the precise forms disclosed.

Provided herein are pressure sensitive adhesive (PSA) formulations and articles of manufacture incorporating same. The disclosure further demonstrates a methodology for selecting PSA formulations and solvent blend combinations that are compatible with long side chain acrylate and methacrylate polymer additives. These selected PSAs and associated solvent systems do not require alteration of the PSA solvent mixture to achieve solubility of the temperature sensitive additive. In addition, the disclosure provides temperature sensitive additives that reduce the adhesion of the PSA to the skin at or above a well-defined temperature.

Further, on-demand removal of pressure sensitive adhesives (PSA) requires additives that reduce bond strength upon exposure to an external trigger. The incorporation of temperature responsive additives into the PSA is a means of introducing thermal debonding behavior into the adhesive interface. These additives can be added directly into the PSA formulation or selectively applied during manufacturing. For skin contact applications, the thermal debonding temperature must not exceed the skin pain threshold temperature (˜45° C.) while remaining above normal skin temperature (˜35° C.). We have identified two classes of materials which possess this targeted temperature behavior: (a) long chain alkanes and their analogous alcohols, and (b) long side-chain acrylate and methacrylate polymers.

Thus, in one aspect, the disclosure provides a pressure-sensitive adhesive (PSA) composition comprising:
(1) a polymeric pressure-sensitive adhesive component, and
(2) a temperature responsive additive to the polymeric PSA component.

In some embodiments, the temperature responsive additive is a crystalline polymeric additive selected from methacrylate or acrylate polymer comprising monomeric units of formula I:

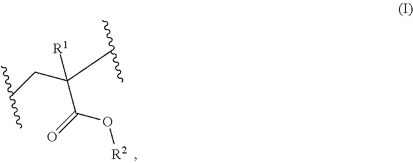

wherein R¹ is H or CH₃, and R² is a C₁₂-C₂₀ linear alkyl.

In some embodiments, the polymeric pressure-sensitive adhesive component is an acrylic-rubber hybrid.

In some embodiments, the temperature responsive additive is dispersed upon the surface of the polymeric PSA component in a plurality of phase separated domains.

In some embodiments, the polymeric pressure-sensitive adhesive composition is solvent borne.

The compositions disclosed herein can comprise other components. In some embodiments, the compositions do not comprise Lanolin, polybutene, or a combination thereof.

In some embodiments, the composition comprises about 0.1 wt % to about 6 wt %, about 1 wt % to about 5 wt %, or about 1 wt % to about 3 wt % of the crystalline polymeric additive. In some embodiments, the composition comprises about 0.5 wt % to about 1 wt % of the crystalline polymeric additive. In some embodiments, the composition comprises about 1 wt % to about 3 wt % of the crystalline polymeric additive. In some embodiments, the crystalline polymeric additive is a copolymer. In some embodiments, the crystalline polymeric additive is a copolymer of C14-alkyl acrylate/C18-alkyl acrylate. In some embodiments, the crystalline polymeric additive is a copolymer of C14-alkyl acrylate/C18-alkyl acrylate with the mole fraction of C14-alkyl acrylate in the copolymer of about 10% to about 50%, about 10% to about 40%, or about 10% to about 35%.

As used herein, the term “about” indicates the subject value can be modified by up to 5% (plus or minus) and remain within the disclosed embodiments.

In some embodiments, the composition is biologically inert, e.g. can be applied to human skin without causing irritation or adverse reactions.

In some embodiments, the composition has a lower adhesive peel force at an elevated temperature than a comparable composition which does not comprise the crystalline polymeric additive. In some embodiments, the composition has a lower adhesive peel force at an elevated temperature than at a human body temperature, e.g., temperatures of about 35° C. to about 45° C. or about 36° C. to about 43° C. In some embodiments, the elevated temperature is a temperature greater than about 40° C., or a temperature of about 40° C. to about 55° C., about 40° C. to about 50° C., or about 40° C. to about 45° C. In some embodiments, the elevated temperature is a temperature greater than about 35° C. to about 55° C.

In some embodiments, the temperature responsive additive covers from 0.1 to 90% of the PSA component. In some embodiments, the temperature responsive additive, upon application of heat to the PSA composition, increases the area of the surface of the polymeric PSA component it covers.

In some embodiments, the crystalline polymeric additive forms crystalline phase separated nanometer-sized domains. In some embodiments, the nanometer sized domains exist on the surface of the adhesive layer. In some embodiments, up to or greater than 20% of the surface is populated by the nanometer sized domains.

In some embodiments, the nanometer-sized domains are 20 to 400 nm. In some embodiments, the nanometer-sized domains may be 20 to 250 nm. In some embodiments, the nanometer sized domains are 20 to 200 nm. In some embodiments, the nanometer-sized domains may be 20 to 50 nm. In some embodiments, the nanometer-sized domains may be from 1 to 999 nm. In other embodiments, the nanometer-sized domains may be up to 10 micrometers.

In another aspect, the disclosure provides an article of manufacture comprising an adhesive composition disclosed herein. Any suitable article of manufacture can incorporate the adhesive composition disclosed herein, such as a tape, a bandage, a wound closure, a wound dressing for use in the medical or surgical field, surgical drapes, veterinary medicine, home pet care, protective flooring, clothing or undergarment, sports equipment, incorporated into a wearable sensor, attachment to a low-surface-energy polyolefin surface such as polyethylene and polypropylene, water immersion application, electronic component, sign for use on a car or window, component in aerospace manufacture, or a label. In some embodiments, the articles of manufacture are intended to be used on human skin. In some embodiments, the articles of manufacture, such as bandages or wound closures, adhere to human skin at a typical human body temperature but can be easily removed without causing pain or irritation at an elevated temperature, for example, at a temperature greater than about 35° C., or a temperature of about 35° C. to about 55° C., about 40° C. to about 50° C., or about 40° C. to about 45° C. In some embodiments, the bandages, tapes, etc. comprising the adhesive compositions do not require application of a release solvent for removal from human skin.

In another aspect, a temperature switchable tape comprising a tape backing layer and an adhesive layer is disclosed. In some embodiments, the adhesive layer further comprises a temperature sensitive additive. In some embodiments, the peel force drops 60% or more upon exposure to temperature up to 45° C. In some embodiments, the peel force of the switchable tape drops 60% or more upon exposure to temperature up to 55° C. In some embodiments, the peel force is measured by the force applied to remove the tape.

In another aspect, the disclosure provides method of reducing the adhesive peel force at elevated temperatures of a solvent borne adhesive composition comprising an acrylic-rubber hybrid, the method comprising:
adding to the solvent borne adhesive composition about 0.1 wt % to about 6 wt % of a crystalline polymeric additive to the composition, wherein the crystalline polymeric additive is selected from methacrylate or acrylate polymer comprising monomeric units of formula I:

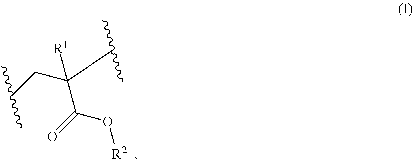

wherein R¹ is H or CH₃, and R² is a C₁₂-C₂₀ linear alkyl. In some embodiments, the acrylic-rubber hybrid is an acrylic polymer copolymerized with a rubber macromer.

In some embodiments, the adding is directly into the solvent borne PSA formulation which allows facile miscibility of the crystalline polymeric additive with the solvent borne adhesive composition.

In some embodiments, the solvent mixture comprises one or more polar solvent and one or more non-polar solvents. In some embodiments, the solvent mixture comprises toluene, heptane, hexane, ethyl acetate, and isopropyl alcohol. In some embodiments, the solvent mixture has a Hansen δ_{P} below about 4.3 MPa^{1/2} and δ_{H} below about 9.1 MPa^{1/2}. In some embodiments, the solvent mixture has a Hansen δ_{P} below about 4.1 MPa^{1/2} and δ_{H} below about 9.1 MPa^{1/2}. In some embodiments, the solvent mixture has a δ_{P} below about 4.1 MPa^{1/2}. In some embodiments, the solvent mixture has a Hansen δ_{P} less than 4.34 MPa^{1/2} and δ_{H} less than 9.12 MPa^{1/2}, wherein the crystalline copolymer of C14/C18 (27%/73%) is added at 1 wt % to the adhesive composition.

In some embodiments, the method further includes evaporating the solvent so that the crystalline polymer additive migrates to the surface of the composition and aggregating the long side chain polymer additive with itself to form phase separated crystalline nanometer-sized domains.

In some embodiments, the solvent borne adhesive composition and the crystalline polymeric additive are dried to influence the size of the crystalline nanometer-sized domains. In some embodiments, the solvent borne adhesive composition and the crystalline polymeric additive are dried at a temperature between 35° C. to 150° C. In some embodiments, the solvent borne adhesive composition and the crystalline polymeric additive are dried for a duration between 1 second and 2 hours. In some embodiments, the solvent born adhesive composition and the crystalline polymeric additive are dried at 120° C. for 10 minutes.

In another aspect, a method of using the composition method includes applying heat to the composition and removing the composition from a surface is disclosed. In some embodiments, the surface is human skin. In some embodiments, the surface is a window.

In another aspect, the disclosure provides a pressure-sensitive adhesive (PSA) composition comprising:
(1) A polymeric pressure-sensitive adhesive component,
(2) A temperature sensitive additive selected from methacrylate or acrylate polymer, and
   wherein the temperature sensitive additive aggregates with itself to form crystalline phase separated nanometer-sized domains.

In some embodiments, the PSA is solvent borne. In some embodiments, the temperature sensitive additive is a long side-chain crystalline polymeric additive.

In some embodiments, the nanometer-sized domains are 1 nm to 10 microns. In some embodiments, the nanometer-sized domains are 20-400 nm.

In another aspect, the disclosure provides a method of making a pressure sensitive adhesive composition, the method comprising mixing a solvent-borne PSA with a temperature responsive additive; applying the mixed adhesive and additive to a surface to be adhered; and drying the adhesive.

In another aspect, the disclosure provides a method of making a temperature-switchable adhesive, the method comprising applying a PSA to a surface (backing) to be adhered, drying the adhesive, and printing a temperature responsive additive onto the adhesive.

In another aspect, the disclosure provides a method of making a temperature-switchable adhesive, the method comprising any combination of grooving or embossing a first PSA surface; and adding an additional layer of PSA with temperature responsive additives dissolved in a solvent mixture that is sprayed, coated printed, or laid over the first PSA surface.

In some embodiments, the method of making a temperature switchable adhesive is further integrated into a roll-to-roll manufacturing setup and/or multi-layer coating process.

One aspect of the invention is based on the fortuitous match of the Hansen Solubility Parameter (HSP) values for rubber-acrylic pressure Sensitive Adhesives (PSA) formulations and polymer additives which can reduce the peel force of the PSA from human skin and other substrates upon heating. The invention employs an additive that consists of long side-chain alkyl acrylate or methacrylate polymers and copolymers. The number of methylene units in the side-chain polymers controls the melting point of these “comb-shaped” materials. When the ambient temperature exceeds the melting point of the additive, the peel force of the PSA is significantly decreased.

Pressure Sensitive Adhesives (PSA) contain a blend of solvents that solubilize the monomer and polymer solutes in the formulation. The solvents are selected based on the chemical nature of the constituent solutes which may possess alkyl, hydroxy, carbonyl functionality. PSAs have been developed that consist of rubber-acrylic hybrid polymers. These rubber-acrylic hybrid PSAs consist of an acrylic polymer backbone grafted with rubber macromers such as ethylene-butylene macromers as described in U.S. Pat. No. 6,642,298. The inventors discovered that solvent borne, hybrid PSAs have solubility characteristics that are remarkably close to the temperature sensitive polymer additives. Therefore, without wishing to be bound by theory, the solvent blends incorporated into the rubber-acrylic PSAs will also provide good solubility of the temperature sensitive additive.

Solvent blends are designed to closely match the properties of the PSA components using the well-known Hansen Solubility Parameters (HSP) methodology. The HSP are a set of three numbers that define the way a solvent will interact with other molecules. Each one of the three parameters, δ_{D}, δ_{P} and δ_{H}, corresponds to a type of interaction: London dispersion force, polar force and hydrogen bonding force, respectively. The dispersion force for relatively small sized solvent molecules does not vary very much and is not a strong indicator of solubility. On the other hand, δP and δH, are more indicative of solvent effectiveness for a given solute. Small solvent δP values are required for non-polar solutes and large δH values denote the ability of the solvent to form hydrogen bonds with the solute.

The total HSP is calculated based on formula 1:

δ_{T}=(δ²_{D}+δ²_{P}+δ²_{H})^{1/2} (1)

In a solvent blend the δ_{D}, δ_{P}, and δ_{H} values are calculated from the volumetric concentration (Cᵢ) weighted value of each individual component in the mixture according to formulas 2, 3, and 4.

δ_{D}=C₁δ_{D1}+C₂δ_{D2}+C₃δ_{D3}+ . . . (2)

δ_{P}=C₁δ_{P1}+C₂δ_{P2}+C₃δ_{P3}+ . . . (3)

δ_{H}=C₁δ_{H1}+C₂δ_{H2}+C₃δ_{H3}+ . . . (4)

where the subscripts 1, 2, 3, etc. correspond to the individual solvent component. HSP parameters for common solvents used in PSA formulations are given in Table 1.

TABLE I
Hansen Solubility Parameters for PSA Solvents

| Solvent | δ_{D} | δ_{P} | δ_{H} |
|---|---|---|---|
| Toluene | 18 | 1.4 | 2 |
| Heptane | 15.3 | 0 | 0 |
| Hexane | 14.9 | 0 | 0 |
| Ethyl Acetate | 15.8 | 5.3 | 7.2 |
| Isopropyl Alcohol | 15.8 | 6.1 | 16.4 |

Previously, the solubility of a solute additive in a PSA was predicted based only on the total HSP value (δ_{T}). However, this can be misleading since δ_{T} values for different solvent mixtures may be quite close together, for example less than 10% difference, and yet the resultant miscibility of a non-polar solute may be substantially different.

Crystalline type polymers are desired since their temperature responsive reduction in peel force will take place over a relatively narrow range of temperature. Acrylate and methacrylate polymers have a backbone consisting of carbon-carbon bonds. The long alkyl side chains are connected to the backbone by an ester linkage. Although this ester functionality contains a hydrogen bonding carbonyl (C═O) group, it is mostly shielded from external solvents by the long alkyl side chains (R) extending from the backbone. Nonpolar methylene (—CH₂—) units enable the polymers to form crystalline solids resembling a type of paraffin wax. Therefore, the solubility of the long-side chain acrylate and methacrylate polymers is mostly restricted to nonpolar type solvents such as toluene and alkanes like hexane and heptane. Copolymers consisting of monomeric units with R>12 and R≤20 are suitable additives for temperature responsive PSAs. The crystalline nature of these copolymers will yield sharp melting point transitions.

Solvent mixtures and PSA formulations that allow facile miscibility of the additive with the PSA may be challenging because it imposes an upper limit on the value of the polar component of the Hansen Solubility Parameter of the PSA and solvent blend and it is overlooked in prior technologies. Table II summarizes the weight fraction of the individual components of the solvent blends for three different Henkel PSA formulations. The TSP additive was only soluble in the AH-115 PSA. Warming and extended stirring did not lead to TSP solubility in the other two PSAs. It should be noted that the AH-115 material is an acrylic-rubber hybrid (U.S. Pat. No. 6,642,298) while the other two materials are acrylic PSAs.

TABLE II
Solvent Properties of PSA Formulations and
Compatibility with Non-Polar Additive

| | Duro-Tak | Duro-Tak | Duro-Tak |
|---|---|---|---|
| Solvent Blend | 129NA | 788NA | AH-115 |
| Toluene | 5% | 17% | 26% |
| Heptane | 33% | | 15% |
| Hexane | | | 18.5% |
| Ethyl Acetate | 30% | 83% | 35.5% |
| Isopropyl Alcohol | 32% | | 5% |
| Additive Solubility | Insoluble* | Insoluble* | >10% |

*Mixtures were heated to 25° C. for ~72 hours

The inventors used 24 different solvent blends to narrowly bracket the solubility of the temperature sensitive polymer additive and distill the limiting solubility parameters. Blends were prepared that matched the composition of the PSA solvents and many additional mixtures extended the range of the HSP values. In the following Table III solubility of the additive in solvent blends (Table V) was established by the absence of turbidity in the mixtures of solvents and additive. Insolubility was established after mixtures were heated to 25° C. for-672 hours. Miscibility of the TSP with the solvent blends were deemed “metastable” (SB-09, SB-14 and SB-16) as they were able to solubilize the TSP upon mixing, but the mixture would become cloudy over a period of time if left undisturbed.

TABLE III
Solvent Blend Measurements
of C14/C18 Additive Solubility

| Solvent | | | | | | |
|---|---|---|---|---|---|---|
| Mixture | Solubility | δ_{D} | δ_{P} | δ_{H} | δ_{T} | Notes |
| SB-01 | Soluble | 16.04 | 2.30 | 3.56 | 16.58 | |
| SB-02 | Soluble | 16.51 | 2.36 | 3.67 | 17.08 | |
| SB-03 | Soluble | 15.86 | 2.37 | 4.82 | 16.74 | |
| SB-04 | Soluble | 16.13 | 2.55 | 3.9 | 16.79 | |
| SB-05 | Soluble | 15.71 | 3.38 | 7.18 | 17.60 | |
| SB-06 | Soluble | 17.07 | 3.39 | 8.10 | 19.20 | |
| SB-07 | Soluble | 15.75 | 3.61 | 7.51 | 17.81 | |
| SB-08 | Soluble | 15.78 | 3.68 | 5.86 | 17.23 | |
| SB-09 | Soluble | 16.92 | 3.70 | 9.06 | 19.55 | Metastable |
| SB-10 | Insoluble | 16.90 | 3.75 | 9.20 | 19.60 | |
| SB-11 | Soluble | 16.86 | 3.76 | 8.61 | 19.30 | |
| SB-12 | Soluble | 15.74 | 3.81 | 6.54 | 17.47 | |
| SB-13 | Insoluble | 16.85 | 3.87 | 9.55 | 19.75 | |
| SB-14 | Soluble | 16.70 | 4.04 | 8.82 | 19.31 | Metastable |
| SB-15 | Insoluble | 16.65 | 4.13 | 9.12 | 19.43 | |
| SB-16 | Soluble | 16.39 | 4.29 | 6.13 | 18.01 | Metastable |
| SB-17 | Insoluble | 16.44 | 4.34 | 7.62 | 18.63 | |
| SB-18 | Insoluble | 16.46 | 4.37 | 8.40 | 18.99 | |
| SB-19 | Insoluble | 16.49 | 4.38 | 9.04 | 19.31 | |
| SB-20 | Insoluble | 16.48 | 4.43 | 9.52 | 19.55 | |
| SB-21 | Insoluble | 16.19 | 4.61 | 6.29 | 17.97 | |
| SB-22 | Insoluble | 15.88 | 4.50 | 7.86 | 18.28 | |
| SB-23 | Insoluble | 15.94 | 4.56 | 9.51 | 19.11 | |
| SB-24 | Insoluble | 16.17 | 4.64 | 6.32 | 17.97 | |

In some embodiments. δP must be less than about 4.34 Mpa^{1/2} and δH must be less than 9.12 Mpa^{1/2} for solubility of the crystalline copolymer of C14/C18 (27%/73%) at 1 wt %. Therefore, a large hydrogen bonding solubility parameter, δH must be taken into consideration for determining TSP solubility based on the solvent mixture measurements. More polar environments are indeed encountered in solvent-based acrylic-only PSA formulations as shown in Table IV.

Most noteworthy is the additive solubility in the solvent mixtures SB-06 and SB-11, where δ_{T} exceeds 19 Mpa^{1/2}, respectively. However, δ_{P} for SB-11 is 3.76 and 3.39 for SB-6 and the additive is soluble. By conventional wisdom, this large δ_{T} value should inhibit solubility. Prior art ('035) set a range of δ_{T} of −0.5 to +1.5 for solubility of the long side-chain polymers in PSA solvents. However, the data in Table III shows that the &T range is closer to +1.5 provided that the δ_{P} and δ_{H} criteria are satisfied. In some embodiments, the δ_{T}±1.5 guideline does not provide solubility since the δ_{P} and δ_{H} criteria are not satisfied.

Since AH-115 meets the solubility condition for δ_{P}, it is unnecessary to remove and substitute solvents in the PSA as with existing technologies. Furthermore, AH-115 has the favorable feature of adhesion to low energy surfaces including human skin and is approved for skin contact. Therefore, the combination of the inert, highly crystalline, and non-polar additive and AH-115 meets the regulatory standards for human skin contact unlike the compositions disclosed in the art.

TABLE IV
Hansen Solubility Parameters for Classes of PSA Materials

| PSA Material | δ_{D} | δ_{P} | δ_{H} | δ_{T} |
|---|---|---|---|---|
| Acrylic - OH | 17.2 | 8.7 | 6.5 | 20.3 |
| Functionality | | | | |
| Acrylic —COOH | 16.4 | 5.6 | 6.9 | 18.6 |
| Functionality | | | | |
| Acrylic-Rubber | 15.8 | 1.2 | 5.4 | 16.7 |
| Hybrid | | | | |
| Acrylic-Rubber | 15.7 | 2.3 | 4.8 | 16.6 |
| Hybrid | | | | |

HSP data for two types of acrylic formulations and acrylic-rubber hybrid formulations are given in Table IV. The same solubility criteria as determined for the solvent blends in Table III also apply to the PSA formulations that consist of solvents and the adhesive components. Table IV shows that the δ_{P} values for acrylic-rubber hybrids (1.2 to 2.3 Mpa^{1/2}) are well within the solubility criteria determined from the solvent blend measurements (Table III). On the other hand, δ_{P} values for acrylic PSAs (5.6 to 8.7 Mpa^{1/2}) do not meet the solubility criteria derived from measurements for the C14/C18 additive and solvent blends (Table III). Therefore, the additive will be insoluble or very slightly soluble in typical acrylic PSAs like 129 and 788.

Based on the solvent blend analysis, the δ_{P} value for AH-115, and most likely other rubber-acrylic PSAs, meets the criteria for solubility of the temperature sensitive additive. This unexpected result enables solubility of many different non-polar, temperature sensitive polymers into rubber-acrylic hybrid class PSAs as evident in Table IV. On the other hand, the δ_{P} solubility parameter of the typical acrylic based class of PSAs is much larger than the rubber-acrylic hybrid PSA. Therefore, the acrylic based PSAs will have poor compatibility with the additive as shown in Table II for the Duro-Tak 129NA and 788NA acrylic PSAs.

Furthermore, in some embodiments, the adhesive peel force of AH-115 can be substantially reduced (˜23-44%) at elevated temperatures (43 C) when 1-5% (w/solids w) of the long side-chain crystalline polymer is added to the PSA. Preliminary experiments have shown that the largest change in peel force at elevated temperature takes place with 1% addition of the crystalline polymer. Changing the chemical composition of the polymer additive enables shifting of the release temperature over a wide range (˜30-45° C.) for medical and other applications. The magnitude of the peel force reduction is related to the polymer additive concentration and permits trade-offs between the initial peel force and the temperature induced drop. For some medical applications it may be desirable to increase the initial adhesion properties of a tape or dressing beyond conventional values and provide trauma free removal using the temperature switching properties of the polymer additive. Meeting current product performance of tape adhesives in direct-to-consumer products, such as tape for easing back pain or cosmetic beauty products, may be sufficient if the top is removable in a warm/hot shower.

TABLE V
Solvent Blend Composition (Volume Percentages)

| Solvent | | | | Ethyl | Isopropyl |
|---|---|---|---|---|---|
| Blend | Toluene | Heptane | Hexane | Acetate | Alcohol |
| SB-01 | 23.8 | 17.4 | 22.4 | 31.3 | 5.1 |
| SB-02 | 42.5 | 0 | 25 | 27.3 | 5.2 |
| SB-03 | 20.3 | 0 | 43.2 | 17 | 19.5 |
| SB-04 | 26 | 15 | 18.5 | 35.5 | 5 |
| SB-05 | 4.5 | 37.7 | 0 | 26 | 31.8 |
| SB-06 | 57.6 | 0 | 0 | 0 | 42.4 |
| SB-07 | 5 | 33 | 0 | 30 | 32 |
| SB-08 | 9.5 | 0 | 25.2 | 54.8 | 10.5 |
| SB-09 | 51 | 0 | 0 | 0 | 49 |
| SB-10 | 50 | 0 | 0 | 0 | 50 |
| SB-11 | 48.2 | 0 | 0 | 9.3 | 42.5 |
| SB-12 | 7.5 | 0 | 25 | 50.8 | 16.7 |
| SB-13 | 47.6 | 0 | 0 | 0 | 52.4 |
| SB-14 | 40.7 | 0 | 0 | 18.6 | 40.7 |
| SB-15 | 38.7 | 0 | 0 | 18.6 | 42.7 |
| SB-16 | 26.6 | 0 | 0 | 70 | 3.4 |
| SB-17 | 29 | 0 | 0 | 50 | 21 |
| SB-18 | 30 | 0 | 0 | 40 | 30 |
| SB-19 | 31.3 | 0 | 0 | 31 | 37.7 |
| SB-20 | 31.1 | 0 | 0 | 26.1 | 42.8 |
| SB-21 | 17.6 | 0 | 0 | 82.4 | 0 |
| SB-22 | 7.8 | 6.2 | 6.4 | 58.1 | 21.5 |
| SB-23 | 9.5 | 13.2 | 0 | 36.5 | 40.8 |
| SB-24 | 17 | 0 | 0 | 83 | 0 |

Mechanisms for the assisted adhesive release include the softening or melting or decreased viscosity of additive domains at the adhesive skin interface leading to the inability for those regions to support peel forces leading to an effective loss of adhesion of those skin facing domains, thereby reducing the overall force required for adhesive removal and the melting or increased surface mobility of the additive domains such that the additives appear at significant areas of the adhesive/skin interface leaded to reduction in adhesion and delamination. In some embodiments, melting or increased surface mobility of the additive domains includes the possibility of the formation of an additive rich layer that is able to more rapidly form at the skin/adhesive interface as well as the spreading and/or modification of additive domains at the adhesive/skin interface leading to a lower angle boundary between the adhesive/domain and skin.

C20 (Eicosane) and C22 (Docosane) alkanes exhibit melting temperatures in the range of 38′C to 43° C. Furthermore, in some embodiments, these alkanes can be combined into an alloy with a melting temperature that is linearly related to the mole ratio of the two alkanes. Hence the melting temperature can be finely tuned to virtually any temperature between that of the two pure compounds. The alkanes are soluble in a solvent based, apolar or non-polar PSA (such as hybrid acrylic-rubber PSA). Tetradecanol is a long chain (C14) alcohol similar in PSA solubility to the alkanes and has a suitable melting temperature (38° C.).

Atomic Force Microscopy (AFM) tapping mode phase and topography images of a solvent borne PSA containing the long-chain alkanes and alcohols did not exhibit surface macrophase separated domains which were clearly seen with a temperature-sensitive polymer (TSP, a C14/C18 copolymer). Furthermore, mixtures of the hybrid PSA with the alkane additive did not show a strong temperature dependent reduction of peel force compared to 1% TSP additive made with 27% C14 and 73% C18 as a copolymer. Based on the AFM images and peel force measurements it appears that the long chain alkanes remain miscible in the PSA formulation following solvent extraction. To overcome this obstacle, a sufficiently high concentration of temperature responsive alkanes at or near the PSA surface so that elevated temperature can disrupt the adhesion to a substrate like skin is disclosed. Several methods may accomplish this, such as adding an additional layer of PSA with temperature responsive alkanes dissolved in a solvent mixture that is sprayed, printed, or laid over native PSA surface or after a mechanical disturbance like grooving or embossing the PSA surface. These methods may be integrated into a roll-to-roll manufacturing process and multi-layer coating arrangement.

In some embodiments, other methods may be used, such as surface imbibing (SI) where the long chain alkanes are applied to the PSA surface following thermal solvent evaporation. The SI approach may use a compatible solvent (e.g., toluene, hexane, heptane) for the long chain alkanes that is also mildly soluble in the surface of the PSA. Solvent immersion swelling of the PSA permits permeation of the solvent borne alkane into the outer surface region. When the SI solvent is subsequently evaporated, the long chain alkane molecules will remain embedded into the outer surface of the PSA. The solvent swelling-impregnation technique has been applied to a hemocompatibility treatment for silicone tubing to inhibit blood coagulation, surface fouling, and inflammation.

In some embodiments, solvent vapor rather than immersion can also lead to swelling of the PSA. In this instance a layer of the long-chain alkane or long-chain alcohol additive is deposited onto the outer surface of the adhesive and the two layers are exposed to the solvent vapors. The solvent must have a high enough vapor pressure (˜100 torr) at ambient or slightly elevated temperature to rapidly swell the outer surface of the PSA coating. In addition, the solvent must afford good solubility for the additives. Two readily available solvents that meet all of these criteria are hexane and cyclohexane.

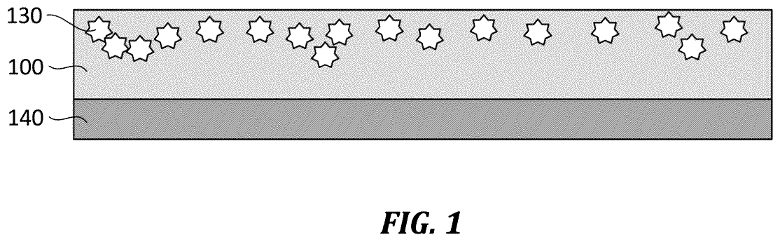

FIG. 1 is an illustration of an example adhesive composition in accordance with the present technology. The adhesive composition may include a substrate 140, a PSA/solvent mixture 100, and temperature sensitive polymer (TSP) nanometer-sized domains 130.

In some embodiments, the substrate 140 is a PET substrate. In other embodiments, the substrate 140 is a thin polyurethane film In some embodiments, the PSA/solvent mixture 100 is AH-115. In some embodiments, the PSA/solvent mixture 100 is applied to the substrate 140 in a thin layer. In some embodiments, the thin layer of PSA/solvent mixture 100 is 50 microns thick.

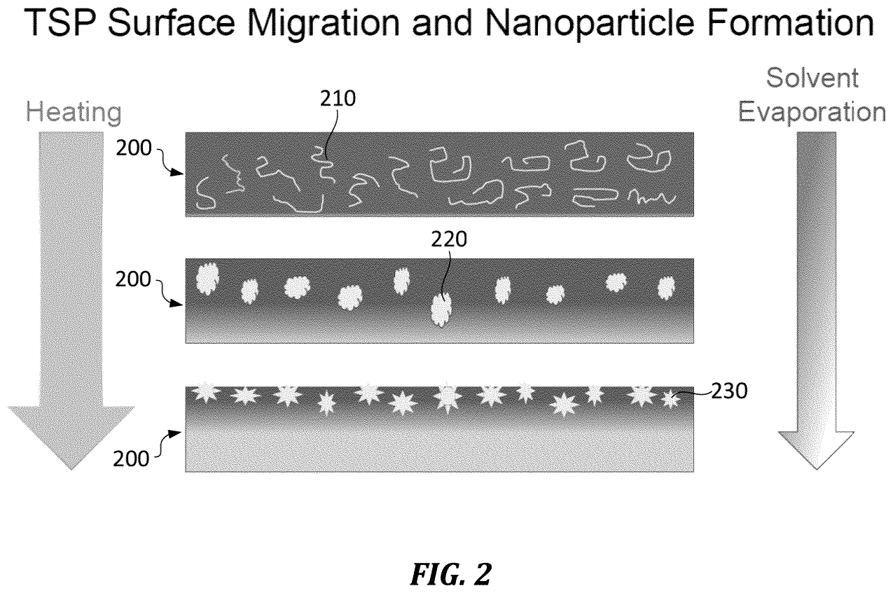

In some embodiments, the nanometer-sized domains 130 are formed through evaporation of the solvent, as illustrated in FIG. 2. In some embodiments, the nanometer-sized domains 130 are 1 to 99 nanometers in diameter. In other embodiments, the nanometer-sized domains 130 are up to 10 microns in diameter. When heated, the presence of the nanometer-sized domains reduces the peel force of the adhesive composition, to remove it more easily after it has been applied to a surface.

FIG. 2 is an illustration of the formation of example nanometer-sized domains in an example adhesive composition in accordance with the present technology. In an embodiment, a PSA/solvent mixture is prepared as part of an adhesive composition. In some embodiments, the PSA/solvent mixture is AH-115. In some embodiments, as illustrated in FIG. 1, the PSA/solvent mixture is applied as a thin film 200 to a substrate (not pictured in FIG. 2). In some embodiments, the PSA/solvent mixture is an adhesive layer on top of a backing surface (not shown in FIG. 2). In some embodiments, the substrate is a PET substrate. In some embodiments, the thin film 200 is 50 microns thick.

When the thin film 200 is wet, polymer molecules, such as illustrated temperature sensitive polymer (TSP) molecules 210 are free to move about within the thin film 200 and may not have any particular orientation or large-scale agglomeration. In some embodiments, TSP molecules 210 are a small amount of additive that is miscible in the PSA/solvent. In some embodiments, the TSP molecules 210 are a copolymer. In some embodiments, the TSP molecules 210 are a combination of a copolymer and another additive. In some embodiments, the TSP molecules 210 are a 1% (w/w % solids) copolymer of C18 and C14. In some embodiments, the copolymer is 73% C18 and 27% C14. In some embodiments, the melting temperature of the TSP molecules 210 is 41° C. In some embodiments, the molecular weight of the TSP molecules 210 is 138.000 Dalton.

In some embodiments, as the thin film 200 is heated, as indicated by the arrow pointing down on the left side of FIG. 2, the solvents in the PSA/solvent mixture begin to evaporate, as indicated by the arrow pointing down on the right side of FIG. 2. In some embodiments, the thin film 200 is heated in an oven. In some embodiments, the oven temperature is set to 120° C. As the solvents in the PSA/solvent mixture begin to evaporate, the TSP molecules 210 migrate and phase separate to the surface of thin film 20, forming small TSP aggregates 220. In some embodiments, the TSP aggregates 220 are more soluble in the solvent phase of the PSA/solvent mixture and therefore tend to migrate along with the evaporating solvents toward the outer, air exposed thin film 200 surface.

In some embodiments, thin film 200 may be dried. As thin film 200 dries, the TSP aggregates 220 form well organized crystalline nanometer-sized domains 230. In some embodiments, the nanometer-sized domains 230 are around 20 to 200 nanometers in diameter. In other embodiments, the nanometer-sized domains 230 can range from 1 nanometer to 10 microns in diameter. In other embodiments, the nanometer-sized domains 230 can be 1 to 99 nanometers in diameter. In some embodiments, the nanometer-sized domains cover up to or greater than 20% of the surface of the thin layer 200.

The lower the concentration of TSP molecules 210, the lower the density of the nanometer-sized domains 230. In some embodiments, the TSP molecules 210 are added to the thin film 200 in a concentration of 1%. In some embodiments, this concentration may be lower, such as 0.01% or 0.05%. The combination between the nanometer-sized domains 230 and the thin film 200 may lead to an 80% reduction in adhesion when heated.

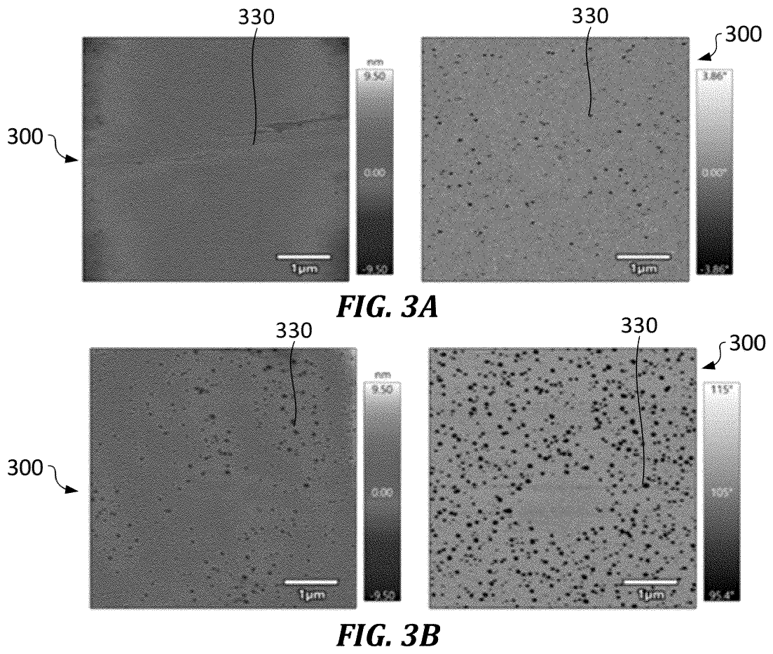

FIGS. 3A-3B are representative tapping mode atomic force microscopy (AFM) images of example nanometer-sized domains in accordance with the present technology. FIGS. 3A-B include a PSA/solvent mixture 30) and TSP additive domains 330. On the left of FIG. 3A is a topography image of an example nanometer-sized domains, and on the right of FIG. 3A is a phase image of the same. On the left of FIG. 3B is a topography image of another example of nanometer-sized domains, and on the right is a phase image of the same. On the right side of the topography images is the topography in nanometers. On the right side of the phase images, is the phase in degrees. On the bottom left of FIGS. 3A-B is a 1 μm scale.

FIGS. 3A-B were achieved with an AFM in tapping mode at RT, a tune amplitude of 300 kHz, a 40 N/m spring constant that is uncalibrated, a free amplitude of 300 mV, a setpoint of 221 mV, a 0% target percent tune offset, and a 0.5 Hz scan rate

In some embodiments, such as shown in FIG. 3A, 0.25% of TSP to PSA/solvent mixture is applied to a 50 micrometer PET backing and dried at 120° C. for 10 minutes. In some embodiments, such as FIG. 3B, 1% of TSP to PSA/solvent mixture is applied to a 50 micrometer PET backing. The additive domains 330 grew in size and number per unit area at the surface with increased TSP concentration. As shown in FIGS. 3A-3B, the additive domains 330 may be well above the volume fractions of the additive. In other embodiments, the concentration of TSP molecules is lower, such as 0.01% or 0.05%. In some embodiments the additive domains 330 are 1 to 200 nanometers in diameter. In other embodiments, the additive domains 330 may range from 1 nanometer to 10 microns in diameter.

In some embodiments, surface segregation and surface domain formation could be enhanced during or after deposition by addition or removal of solvent vapor to the atmosphere. Solvent removal could allow faster phase separation and can act from the top surface. Solvent could also be sprayed on to the top surface to promote surface segregation of adhesive domains to the top (ultimately skin facing) adhesive surface.

In some embodiments, multi-solvent adhesive added to additive coating or printing solutions comprising solvents with different vapor pressures at the deposition or post deposition thermal treatment stages and/or different solubilities for the additive and the adhesive also present options to apply additive to PSAs. In the case of differing solvent vapor pressures, the higher vapor pressure solvent will leave the film more rapidly after deposition and/or thermal treatment of the additive, adhesive, and solvents mixture. This may result in a solidified or gelled film consisting predominantly of the additive, adhesive and the lower vapor pressure solvent. In this case the residual lower vapor pressure solvent assists in the segregation of the additive to the skin/adhesive interface. That lower vapor pressure solvent can then be removed by evaporation from the film over time and/or at higher temperatures. The differential solubility in solvent-blend based coating or printing compositions may promote the precipitation, gelling or solidification of one phase before the other, creating an advantageous additive-segregated structure.

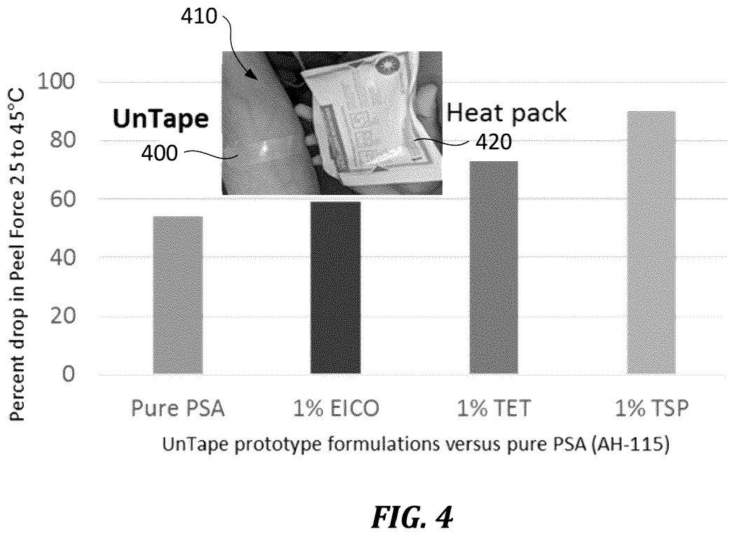

FIG. 4 is a graph of reduction in adhesion measured using a temperature-controlled peel force tester. Formulations representing pure PSA and various temperature-responsive additives, in accordance with the present technology. On the x-axis of the graph is pure PSA (AH-115 from Henkel Corporation) using the same 50-micron thick polyethylene terephthalate (PET) clear backing as illustrated in FIG. 2 compared to example additives, EICO (Eicosane at 1% by weight), TET (Tetradecanol at 1% by weight), and TSP (Temperature-Sensitive Polymer of C14 and C18 at 1% by weight). On the y-axis is the percent drop in peel force when the tape was subjected to increased temperatures from 25 to 45° C.

As shown, in FIG. 4, the peel force drops significantly more when additives are added compared to the Pure PSA than any other additive, with 1% TSP molecules resulting in the largest drop in peel force at 45° C.

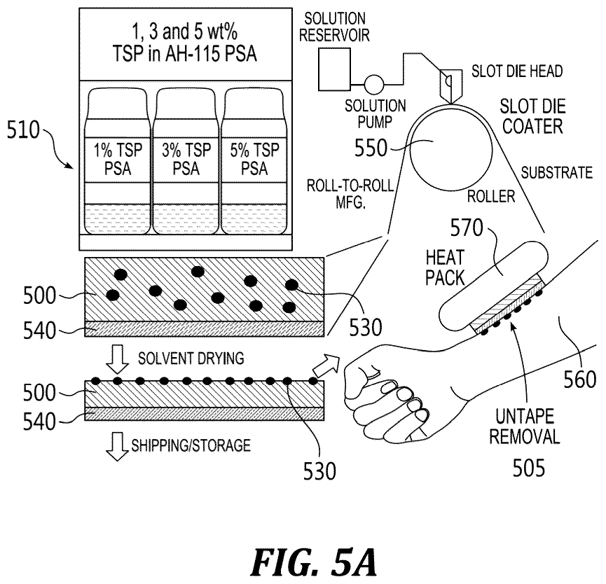

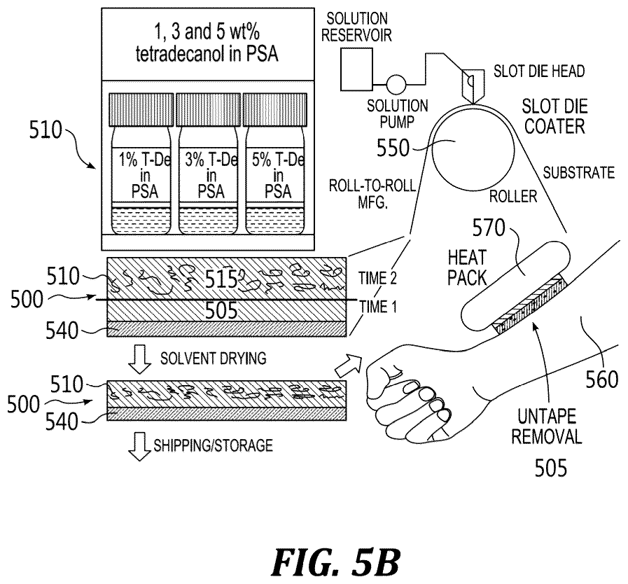

A photograph of the TSP prototype tape 400 is shown on a human forearm 410 with a heat pack 420 which can be used for reducing adhesion immediate before removal. FIG. 1 (insert). In some embodiments, the prototype tape 400 is fabricated using the protocol for fabrication illustrated in FIGS. 5A-B.

FIGS. 5A-B are illustrations of example fabrication processes of an example temperature-responsive medical tape “UnTape”, in accordance with the present technology.

In FIG. 5A, 1%, 3%, and 5% TSP (additive molecules) 510 are added to a PSA/solvent mixture 500. The additive molecules 510 are fully miscible in the PSA/solvent mixture 500. In some embodiments, the additive molecules 510 may be TSP. The PSA/solvent mixture 500 may be applied in a layer onto a substrate 540. In some embodiments, the substrate 540 may be a backing to an adhesive. In some embodiments, the adhesive composition (UnTape) 505 can be applied to a surface 560, and removed with a heat pack 570. In some embodiments, such as the one illustrated in FIGS. 5A-B, the surface 560 is human skin. In other embodiments, the surface 560 may be animal skin, surgical drapes, cars, windows, flooring, clothing or undergarments, sports equipment, sensors, electronic components, and aerospace manufacture components, among others.

In some embodiments, an additive 510, such as TSP, is dissolved in the nonpolar solvent and PSA/solvent mixture 500, which exhibits miscibility by having a clear solution of up to 5% in relative weight of TSP and PSA (top left photograph). In some embodiments, the PSA/solvent mixture 500 is an hybrid acrylic-rubber AH-115 PSA in solvent (Henkel Corporation). In some embodiments, the additive 510 is tetradecanol that is dissolved in the nonpolar solvent+PSA mixture, which exhibits miscibility by having a clear solution of up to 10% in relative weight of tetradecanol and PSA.

As the PSA/solvent mixture 500 dries, the additive 510 may aggregate into phase-separated domains (nanometer-sized domains) 530 and populate the surface of the PSA/solvent mixture 500. In some embodiments, the PSA/solvent mixture 500 is dried in a solvent drying oven at 120° C. for 10 minutes, but it may be dried at any controlled temperature that enables solvent evaporation. In some embodiments, the composition may be dried at another temperature and/or for a longer or shorter duration. In some embodiments, the temperature, time, or partial pressure of the solvent drying oven influences the size of the nanometer-sized domains 530.

In FIG. 5B, the additive molecules 510 are tetradecanol. In some embodiments, tetradecanol can be added to the PSA/solvent mixture in concentrations of 1%, 5%, and 10%. In some embodiments, a base layer of PSA/solvent mixture 505 without additive 510 can be placed directly on substrate 540 at Time 1, and a second layer 515 of PSA/solvent mixture with the additive 510 in it may be placed on top of the base layer at Time 2. In operation, this method creates a higher population of additive 510 at the surface of the PSA/solvent mixture 500 without needing to rely on additive 510 migration to the surface. The additive 510 may not aggregate to form nanometer-sized domains 530 in this method.

In some embodiments, the adhesive mixture may be transferred to a slot die coater 550 and applied onto a backing surface. In some embodiments, the backing surface may be a pre-cleaned plastic backing surface.

In one embodiment, a solid additive 510 such as TSP or tetradecanal is added to PSA and suspended in a solvent. The mixture is then stirred for 24 hours at room temperature. Next, the mixture is transferred to a slot die coater 550 located in a humidity and temperature-controlled room to form a film. In some embodiments, the film consists of a first layer 505 placed directly on the substrate at Time 1 that contains no additive 510, and a second layer 515 placed on top of the first layer 505 that contains additive 510 at Time 2. The film is then applied onto a plastic backing surface that has been pre-cleaned using UV and ozone treatment. The composition is then placed in a solvent drying oven at 120° C. for 10 minutes but may be dried at any controlled temperature that enables solvent evaporation. The composition is then transferred to a release liner for storage or for transfer onto another tape backing material. The tape characteristics can then be measured to verify dried film thickness and peel-force reduction when exposed to elevated temperature. In some embodiments, the PSA surface layers are analyzed to determine the concentration of temperature responsive additives in or on the PSA coating.

Although slot die coating has been shown in FIG. 5B as a means for creating a base PSA layer 505 and then a temperature responsive second PSA layer 515, other coating and printing approaches are possible. The layers could be coated by other techniques such as screen printing, roll coating, gravure, dip coating or spray coating. Spray coating could also be used to coat a base layer with the temperature responsive additive such that the additive segregates into domains at the surface. Patterning the release additive directly on to the adhesive is achieved by a printing process such as flexography, inkjet printing, offset printing, gravure, electrohydrodynamic printing, or screen printing. This could be done in roll or sheet formats.

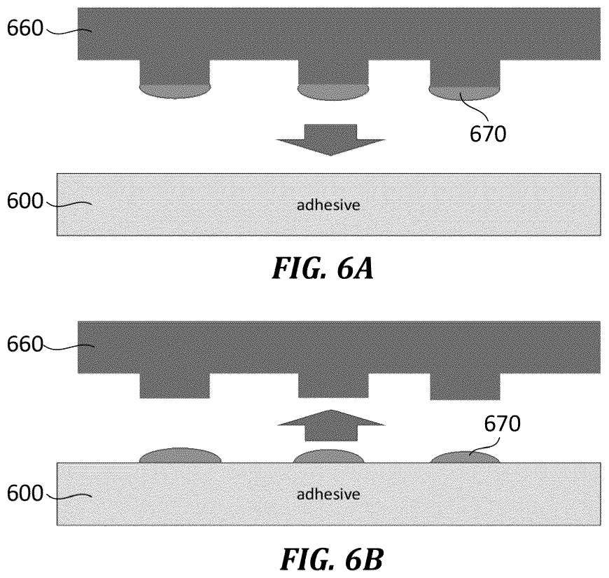

FIGS. 6A-B are diagrams of a flexographic printing plate transferring additive ink domains to the adhesive surface in a defined pattern in accordance with the present technology. FIGS. 6A-B are not to scale. FIGS. 6A-B include a flexographic plate 660 with additive ink 670, and an adhesive composition 600.

FIG. 6A shows flexographic place 660 with additive ink 670 applied to it. Flexographic plate 660 is brought towards the adhesive composition 600 as indicated by the arrow pointing down. In FIG. 6B, the flexographic plate 660 has made contact with the adhesive composition 600 and transferred the additive ink 670 onto the adhesive composition 600 in a pattern. The flexographic plate 660 is withdrawn as indicated by the arrow pointing upwards.

In operation, flexography and related techniques may be advantageous as there is not contact made between the printing or deposition mechanism and the adhesive surface 600 except where the additive ink 670 is present, minimizing potentially problematic adhesion between the printing mechanism and the adhesive 600 where additive 670 is not present. Other non-contact printing techniques such as inkjet, aerosol jet or electric field assisted jet printing could also be used. It may also be possible to transfer the additive to the adhesive surface by laser ablation printing in which laser energy, which can be delivered in a controlled pattern, is used to ablate or thermally assist transfer of additive from a carrier film in contact or in near proximity to the adhesive surface.

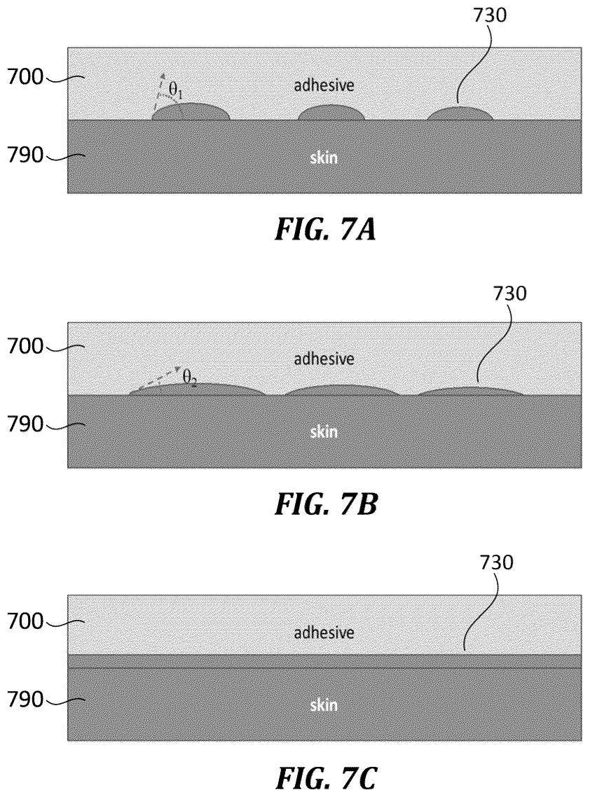

FIGS. 7A-C shows a series in time that describes an example mechanism of release from temperature-responsive domains on the surface of the PSA, which can be in contact with skin in accordance with the present technology. Illustrated in FIGS. 7A-C is an adhesive composition 7M), phase separated (nanometer-sized) domains 730 and a surface (skin) 790. The surface 790 may be a surface including but not limited to human skin, animal skin, surgical drapes, cars, windows, flooring, clothing or undergarments, sports equipment, sensors, electronic components, and aerospace manufacture components.

The adhesive 700 may be exposed to heat. In some embodiments, the heat is between 35 OC and 55° C. In FIG. 7A, the phase-separated domains 730 begin melting, they no longer can support stress and now have a stress concentration point at the perimeter of the domains 730 and reduced peel strength. A contact angle of the phase-separated domains 730 is shown with the illustrated angle θ₁.

In FIG. 7B, the phase-separated domains 730 may increase in mobility and/or change surface energy. The contact angle, θ₂ may decrease in response to the increasing stress concentration and reduction in peel force. The interfacial area of the adhesive 700 to the skin 790 is also reduced.

In FIG. 7C, the phase separated domains 730 may spread to cover the entire interface between the adhesive 700 and the skin 790.

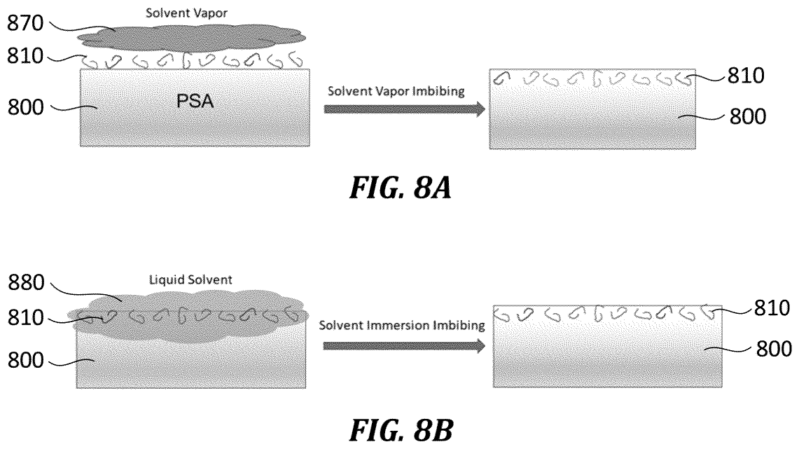

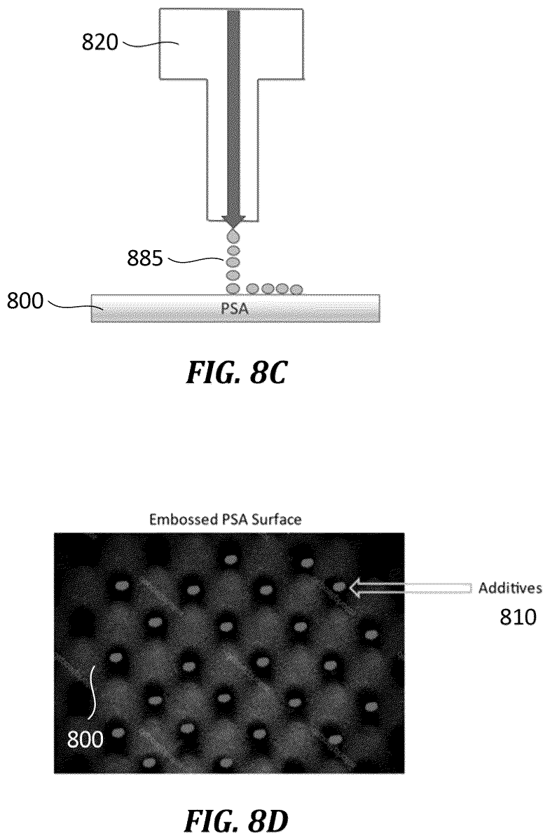

FIG. 8A-D illustrate alternative methods of creating a higher concentration of temperature-responsive additive at or near the surface of the PSA in accordance with the present technology. Additive molecules 810 may be added to a PSA/solvent mixture 800 in a variety of ways, as illustrated in FIGS. 8A-D.

FIG. 8A is an example solvent vapor method. FIG. 88 is an example solvent immersion method. In some embodiments, the solvent can be solvent vapor 870, as shown in FIG. 8A or liquid solvent 880 as shown in FIG. 8B. In some embodiments, prior to solvent vapor 870 exposure, the solid additive particles 810 are deposited onto the PSA surface 800 by means of a stamp, flexography, inkjet printing, offset printing, gravure, electrohydrodynamic printing, or screen printing. In some embodiments, the solvent vapor 870 induces swelling which is conducive to imbibing the additive. In some embodiments, upon imbibing the surface with the additive, the solvent reside is removed from the PSA/solvent mixture 800 by evaporation.

FIG. 8C is an example drop-on demand inkjet printing approach. In some embodiments, the additive 810 is dissolved in a carrier solvent 885, e.g. hexane, and droplets are dispensed through the inkjet nozzle 820. In some embodiments, following the surface deposition, the excess solvent 885 is removed by evaporation. FIG. 8D is an example embossed PSA surface 800 with additive particles 810 in the surface recesses.

While illustrative embodiments have been illustrated and described, it will be appreciated that various changes can be made therein without departing from the spirit and scope of the invention.

1. A pressure-sensitive adhesive (PSA) composition comprising:
(1) a polymeric pressure-sensitive adhesive component, and
(2) a temperature responsive additive to the polymeric PSA component.

2. The composition of claim 1, wherein the temperature responsive additive is a crystalline polymeric additive selected from methacrylate or acrylate polymer comprising monomeric units of formula I: 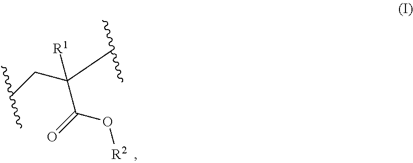
wherein R¹ is H or CH₃, and R² is a C₁₂-C₂₀ linear alkyl.

3. The composition of claim 1, wherein the polymeric pressure-sensitive adhesive component is an acrylic-rubber hybrid.

4. The composition of claim 1, wherein the temperature responsive additive is dispersed upon the surface of the polymeric PSA component in a plurality of phase separated domains.

5. The composition of claim 1, wherein the polymeric pressure-sensitive adhesive composition is solvent borne.

6. The composition of claim 1, wherein the composition does not comprise Lanolin, polybutene, or a combination thereof.

7. The composition of claim 2, wherein the composition comprises about 0.1 wt % to about 6 wt %, about 1 wt % to about 5 wt %, or about 1 wt % to about 3 wt % of the crystalline polymeric additive.

8. The composition of claim 2, wherein the crystalline polymeric additive is a copolymer of C14-alkyl acrylate/C18-alkyl acrylate.

9. The composition of claim 2, wherein the mole fraction of C14-alkyl acrylate in the copolymer is about 10% to about 50%, about 10% to about 40%, or about 10% to about 35%.

10. The composition of claim 1, wherein the composition is biologically inert.

11. (canceled)

12. The composition of claim 1, wherein the composition has a lower adhesive peel force at an elevated temperature than at a human body temperature.

13. The composition of claim 12, wherein the elevated temperature is a temperature greater than about 35° C. to about 55° C.

14. The composition of claim 1, wherein the temperature responsive additive covers from 0.1 to 90% of the surface of the polymeric PSA component.

15. The composition of claim 1, wherein the temperature responsive additive, upon application of heat to the PSA composition, increases the area of the surface of the polymeric PSA component it covers.

16. The composition of claim 2, wherein the crystalline polymeric additive forms crystalline phase separated nanometer-sized domains.

17. The composition of claim 16, wherein the nanometer-sized domains are on a surface of the adhesive layer, wherein the nanometer-sized domains cover up to or greater than 20% of the surface.

18. The composition of claim 16, wherein the phase separated nanometer-sized domains are 1 nanometer to 10 microns.

19. An article of manufacture comprising an adhesive composition of claim 1.

20. (canceled)

21. A switchable tape comprising:
a tape backing layer; and
the pressure sensitive adhesive of claim 1;
wherein the adhesive layer further comprises an adhesive dropping in peel force upon exposure to temperature over 35° C.

22-42. (canceled)

43. A method of making the pressure sensitive adhesive composition of claim 1, the method comprising:
mixing a solvent-borne PSA with a temperature responsive additive;
applying the mixed adhesive and additive to a surface to be adhered; and
drying the adhesive.

44-46. (canceled)
